(19) United States
(12) Patent Application Publication
(10) Pub. No.: US 2004/0086501 A1
(43) Pub. Date: May 6, 2004

(54) Protein having activity as an angiogenesis modulator

(76) Inventor: Gayle Delmonte Wetzel, Martinez, CA (US)

Correspondence Address: Melissa A Shaw, Bayer Corporation, 800 Dwight Way, PO Box 1986, Berkeley, CA, 94701, US

(21) Appl. No.: 10/019,065

(22) PCT Filed: Mar. 30, 2001
(86) PCT No.: PCT/US01/10222

(30) Foreign Application Priority Data
Mar. 31, 2000 (US) 60266300

(51) Int. Cl. A61K 38/48; C12N 9/64

(52) U.S. Cl. 424/94.63; 435/226

(57) ABSTRACT

BTL.012 is a novel human protein useful for regulating or modulating angiogenesis. BTL.012, or variants thereof, may be employed as therapeutics in diseases such as cancer, wound healing, diabetic retinopathies, macular degeneration, and cardiovascular diseases, and other diseases or clinical conditions where angiogenesis is relevant to the causation or treatment of the disease.

### BACKGROUND OF THE INVENTION

1. Field

This invention relates to newly identified polynucleotides, polypeptides encoded by such polynucleotides, the use of such polynucleotides and polypeptides, as well as the production of such polynucleotides and polypeptides. More particularly, the polypeptide of the present invention, hereinafter referred to as BTL.012, has been identified as having a thrombospondin repeat domain and as being active in modulating angiogenesis activity.

2. Background

The thrombospondin family of proteins includes large, multidomain glycoproteins involved in the regulation of proliferation, adhesion, migration and angiogenesis (Mosher, Ann. Rev. Med. (1990) 41: 85-97; Frazier, Curr. Opin. Cell Biol. (1990) 3: 797-99; Bornstein FASEB J. (1992) 6: 3290-99; Lahav, Biochim. Biophys. Acta (1993) 1182: 1-14). The prototype of this family is Thrombospondin-1 (TSP-1), which was first identified as a protein associated with the surfaces of thrombin-stimulated platelets (Baenziger et al., Proc. Natl. Acad. Sci. U.S.A. (1971) 68: 245-49). TSP-1 is a homotrimer with each subunit comprising a 1152 amino acid polypeptide. The complete amino acid sequence of TSP-1 has been determined from cDNA clones isolated by various groups (e.g. Lawler et al. J. Cell Biol. (1986) 103: 1635-48; Kobayashi et al., Biochemistry (1986) 25: 8418-25; and Dixit et al., Proc. Natl. Acad. Sci. U.S.A. (1986) 83: 5449-53). TSP-1 contains a heparin-binding domain, a procollagen homology domain, and three types of repeated domains termed the type 1 (TSP or properdin), type 2 (EGF-like), and type 3 (Ca⁺⁺-binding) repeats (Bornstein, FASEB J. (1992) 6: 3290-99). Five members of the TSP family have been discovered, termed as TSP-1, TSP-2, TSP-3, TSP-4 and COMP/TSP-5 (Bornstein, J. Cell Biol. (1995) 130: 503-06). TSP-1 and TSP-2 are structurally more similar to each other than to TSP-3, TSP4, or TSP-5 (Bornstein and Sage, Meth. Enzymol. (1994) 245: 62-85). Both TSP-1 and TSP-2 are secreted as disulfide-bonded homotrimers whereas TSP-3, TSP-4, and TSP-5 are pentameric. TSP-1 and TSP-2 interact with a number of cell surface receptors, including integrin v-3 LDL-related receptor protein, and heparin sulfate proteoglycans (Chen et al., J. Biol. Chem. (1994) 269: 32226-32; and Chen et al., J. Biol. Chem. (1996) 271: 15993-99).

In vitro and in vivo assays demonstrate that TSP-1 and TSP-2 act both as angiogenesis inhibitors and as potent suppressors of malignant tumor growth (Weinstat et al., Cancer Research (1994) 54: 6504-11; Bleuel et al., Proc. Natl. Acad. Sci. U.S.A. (1999) 96: 2065-70; Streit et al., Am. J. Pathol. 155: 441-52; and Streit et al., Proc. Natl. Acad. Sci. U.S.A. (1999) 96: 14888-93). TSP-1 and TSP-2 are shown to be highly expressed in developing blood vessels, indicating potential roles in primary angiogenesis (Iruela-Arispe et al., Dev. Dyn. (1993) 197: 40-56; and Reed et al., Am. J. Pathol. (1995) 147: 1068-80). Indeed, targeted disruption of the TSP-2 gene significantly increased numbers of small- and mid-sized blood vessels in several tissues including skin (Kyriakides et al., J. Cell Biol. (1998) 140: 419-13).

The regions responsible for inhibition of angiogenesis by TSP-1 have been mapped to the procollagen domain and to the type 1 repeats (Tolsma et al., J. Cell Biol. (1993) 122: 497-511). It is suggested that the inhibition of capillary growth by TSP-1 is multifactorial and involves competition for FGF-2 binding to the endothelial cell surface, binding to heparin sulfate proteoglycans, activation of TGF-beta, and/or binding to CD-36, a receptor for TSP-1 (Vogel et al., J. Cell Biochem. (1993) 53: 74-84; Taraboletti et al., Cell Growth Diff. (1997)\8: 471-79; Schultz-Cherry et al., J. Biol. Chem. (1995) 27: 7304-10; and Dawson et al., J. Cell Biol. (1997)138: 707-17). A truncated TSP subunit was found to both inhibit the proliferation of endothelial cells and to result in increased concentrations of plasminogen activator inhibitor-1, indicating that TSP may affect the process of angiogenesis through at least two mechanisms—proliferation of cells in neovascularization and degradation of the extracellular matrix (Bagavandoss et al., Biochem. Biophys. Res. Comm. (1993) 192: 325-32).

The assays for the antiangiogenesis activity include corneal pocket assay, chorioallantoic membrane angiogenesis assay (CAM), and endothelial cell proliferation and migration assays. Various TSP family proteins, including full length expressed TSP family proteins from transfected mammalian cells, various portions of the repeat domains expressed in bacterial systems, and synthesized peptides, have been used in the above mentioned assays. A recent study has identified two regions of type 1 repeats as potent inhibitors of angiogenesis (Iruela-Arispe et al., Circulation (1999) 100: 1423-31). An N-terminal tryptophan rich domain as well as a C-terminal CSVTCG (SEQ ID NO:5) sequence have been shown to independently inhibit neovascularization. The N-terminal domain showed a stronger inhibition activity against FGF-2-driven angiogenesis, whereas the second region equally blocked the angiogenesis induced by either FGF-2 or VEGF (Iruela-Arispe et al., Circulation (1999) 100: 1423-31).

Recently, a novel brain-specific angiogenesis inhibitor (BAI-1) was identified and cloned (Nishimori et al., Oncogene (1997) 15: 2145-50). It contains 5 TSP type-1 repeats. Recombinant proteins containing these repeats inhibited in vivo neovascularization induced by bFGF in the rat cornea assay. Vascularization is decreased in pulmonary adenocarcinoma expressing BAI-1 (Hatanaka et al., Int J Mol Med. (2000) 5: 181-3). Two BAI-1 homologs have recently been cloned and named as BAI-2 and BAI-3 (Shiratsuchi et al., Cytogenet. Cell Genet. (1997) 79: 103-108). Like BAI-1, BAI-3 was absent or significantly reduced in some glioblastoma cell lines, suggesting that members of this novel gene family may play important roles in suppression of glioblastoma.

Angiogenesis, the formation of new capillaries from preexisting blood vessels, is a multistep, highly orchestrated process involving vessel sprouting, endothelial cell migration, proliferation, tube differentiation, and survival. Several lines of direct evidence now suggest that angiogenesis is essential for the growth and persistence of solid tumors and their metastases (Folkman et al. (1989) Nature 339:58-61; Hori et al. (1991) Cancer Research 51:6180-84; Kim et al. (1993) Nature 362:841-844; Millauer et al. (1996) Cancer Research 56:1615-20). To stimulate angiogenesis, tumors up regulate their production of a variety of angiogenic factors, including the fibroblast growth factors (FGF and BFGF) (Kandel et al. (1991) Cell. 66:1095-104) and vascular endothelial cell growth factor/vascular permeability factor (VEGF/VPF). However, many malignant tumors also generate inhibitors of angiogenesis, including angiostatin and thrombospondin (Chen et al. (1195) Cancer Research 55:4230-33; Good et al. (1990) Proc Natl Acad Sci U S A. 87:6624-28; O'Reilly et al. (1994) Cell 79:315-28). It is postulated that theangiogenic phenotype is the result of a net balance between these positive and negative regulators of neovascularization (Good et al. (1990), supra; O'Reilly et al. (1994), supra; Parangi et al. (1996) Proc Natl Acad Sci U S A. 93:2002-07; Rastinejad et al. (1989) Cell 56:345-55).

Several other endogenous inhibitors of angiogenesis have been identified, although not all are associated with the presence of a tumor. These include platelet factor 4 (Gupta et al. (2000) Blood 95:147-55), interferon-alpha, interferon-inducible protein 10 (Angiolilloet al. (1996) Ann. N.Y. Acad. Sci. 795:158-67; Strieter et al. (1995) J. Biol. Chem. 270:27348-57), which is induced by interleukin-12 and/or interferon-gamma, gro-beta (Cao et al. (1995) J. Exp. Med. 182:2069-77), and the 16 kDa N-terminal fragment of prolactin (Clapp et al. (1999) Invest. Ophthalmol. Vis. Sci. 40:2498-505). The only known angiogenesis inhibitor which specifically inhibits endothelial cell proliferation is angiostatin (O'Reilly et al. (1994), supra). Angiostatin is an approximately 38 kiloDalton (kDa) specific inhibitor of endothelial cell proliferation. Angiostatin is an internal fragment of plasminogen containing at least three of the five kringles of plasminogen. Angiostatin has been shown to reduce tumor weight and to inhibit metastasis in certain tumor models. (O'Reilly et al. (1994), supra).

### SUMMARY OF THE INVENTION

We have now discovered a new protein, hereinafter referred to as BTL.012, which has been identified as having a thrombospondin repeat domain and as being active in modulating angiogenesis activity.

The instant invention encompasses the use of BTL.012 for regulating or modulating angiogenesis. The current invention further encompasses the use of BTL.012 for the treatment of a disease or clinical condition where angiogenesis is relevant to the causation or treatment of the disease or clinical condition, including but not limited to cancer, wound healing, diabetic retinopathies, macular degeneration, and cardiovascular diseases. The instant invention also encompasses pharmaceutical compositions containing BTL.012 and the use of the pharmaceutical compositions for the treatment of the abovementioned diseases or clinical conditions.

In accordance with one aspect of the present invention, there are provided novel mature polypeptides comprising the amino acid sequence given in SEQ ID NO:1 as well as biologically active and diagnostically or therapeutically useful fragments, analogues and derivatives thereof As an additional aspect of the present invention, there are provided antibodies to the polypeptides of the present invention, especially antibodies which bind specifically to an epitope made up of the sequence described in SEQ ID NO:1 or a sequence which shares at least a 60%, preferably at least a 70%, more preferably at least an 80%, still more preferably a 90%, or most preferably at least a 95% sequence identity over at least 20, preferably at least 30, more preferably at least 40, still more preferably at least 50, or most preferably at least 100 residues with SEQ ID NO:1.

In accordance with another aspect of the present invention, there are provided isolated nucleic acid molecules encoding the polypeptides of the present invention, including mRNAs, DNAs, cDNAs, genomic DNA, as well as antisense analogs thereof and biologically active and diagnostically or therapeutically useful fragments thereof.

In accordance with still another aspect of the present invention, there are provided processes for producing such polypeptides by recombinant techniques through the use of recombinant vectors. As a further aspect of the present invention, there are provided recombinant prokaryotic and/or eukaryotic host cells comprising a nucleic acid sequence encoding a polypeptide of the present invention.

In accordance with a further aspect of the present invention, there is provided a process for utilizing such polypeptides, or polynucleotides encoding such polypeptides, for therapeutic purposes, for example, the treatment of cancer, wound healing, diabetic retinopathies, macular degeneration, and cardiovascular diseases.

In accordance with another aspect of the present invention, there are provided nucleic acid probes comprising nucleic acid molecules of sufficient length to specifically hybridize to a polynucleotide encoding a polypeptide of the present invention.

In accordance with yet another aspect of the present invention, there are provided diagnostic assays for detecting diseases or susceptibility to diseases related to mutations in a nucleic acid sequence of the present invention and for detecting over-expression or underexpression of the polypeptides encoded by such sequences.

In accordance with another aspect of the present invention, there is provided a process involving expression of such polypeptides, or polynucleotides encoding such polypeptides, for purposes of gene therapy. As used herein, gene therapy is defined as the process of providing for the expression of nucleic acid sequences of exogenous origin in an individual for the treatment of a disease condition within that individual.

### BRIEF DESCRIPTION OF THE FIGURES

FIG. 1 shows the alignment of various thrombospondin domain type I repeats in Thrombospondin 1 and 2 (TSP1 & 2), Brain-specific Angiogenesis Inhibitor 1, 2, and 3 (BAI1, 2 & 3), and BTL.012. Numbers in the right column reflect the approximate location of the type 1 repeat in the full length protein, or, for BTL.012, the location in SEQ ID NO:33.

FIG. 2 shows the thromospondin domain repeat alignment with brain-specific angiogenesis inhibitor 1. (Query: is SEQ ID NO:31; Sbjct: is SEQ ID NO:32.)

FIG. 3 illustrates the dose dependent inhibitory effect of BTL.012 containing protein supernatants on capillary-like organization of HUVEC cells in MATRIGEL. Results are expressed as percentage of control, which represents the capillary-like organization of untreated HUVEC cells in MATRIGEL.

FIG. 4 illustrates the dose dependent inhibitory effect of purified BTL.012 protein on capillary-like organization of HUVEC cells in MATRIGEL. Results are expressed as percentage of control, which represents the capillary-like organization of untreated HUVEC cells in MATRIGEL.

FIG. 5 illustrates the dose dependent inhibitory effect of purified BTL.012 protein on capillary-like organization of MLuEC cells in MATRIGEL. Results are expressed as percentage of control, which represents the capillary-like organization of untreated HUVEC cells in MATRIGEL.

### SPECIFIC EMBODIMENTS

### Materials and Methods

In vitro MATRIGEL assay: Human umbilical cord endothelial cells (HUVEC, from ATCC, Manassas, Va.) were seeded at 3×10⁴ cells per well in HUVEC complete medium. The HUVEC complete medium contained F12K medium with 2 mM L-glutamine, 100 ug/ml Heparin, 50 ug/ml Endothelial cell growth supplement (ECGS), and 10% fetal bovine serum(FBS). Murine lung endothelial cells (MLuEC) were seeded at 5×10⁴ cells per well in a complete medium containing DMEM with 2 mM L-glutamine, 1% Pen/Strep, and 10% FBS. MATRIGEL basement membrane matrix (Becton Dickinson, Franklin Lakes, N.J.) was prepared using pre-cooled, pipettes, tips, plates and tubes during handling of the matrix. The matrix was thawed at 4° C. overnight on ice, used to coat a 24-well plate (Costar, VWR, West Chester, Pa.) at 0.3 ml/well, and then polymerized at 37° C. for 2 hours. Test samples were added in 0.5 ml of complete medium per well, and cells were added in 0.5 ml of medium per well, so the total volume of medium per well was 1.0 ml. Experiments were conducted in triplicate, with varying dilutions of test samples (from 1:10 to 1:10000) or varying protein concentration (from 100 nM to 1 fM). Cells were incubated overnight at 37° C., 5% CO₂, then fixed and stained using a DIFF-QUIK staining set (VWR, West Chester, Pa.). Plates were dipped in Fixative Solution for 5 seconds, in Solution 1 for 5 seconds, and in Solution 2 for 5 seconds, then rinsed with deionized water and allowed to dry. Plates were then examined under inverted microscope, and quantitative analysis of the capillary-like structures was performed. As used herein, the term “capillary-like structures” includes organized cells in vivo or in vitro leading up to and participating in angiogenesis which results in the cells in association with each other and forming capillaries.

The polypeptides of the present invention include polypeptides having the deduced amino acid sequence given by SEQ ID NO:1. The polypeptides of the present invention may include additional amino acid sequences appended to the N- or C-terminal of the peptides having the deduced amino acid sequence given by SEQ ID NO:1. The polypeptides of the present invention may be recombinant polypeptides, natural polypeptides, or synthetic polypeptides, preferably recombinant polypeptides. As used herein, “protein” is synonymous with “polypeptide.”

The present invention further includes a polypeptide which shares at least a 60%, preferably at least a 70%, more preferably at least an 80%, still more preferably a 90%, or most preferably at least a 95% sequence identity over at least 20, preferably at least 30, more preferably at least 40, still more preferably at least 50, or most preferably at least 100 residues with SEQ ID NO:1. (Such polypeptides may be herein referred to as “polypeptides of the present invention”.) As used herein, a “BTL.012-like protein” means a polypeptide of the present invention as referred to in this paragraph. A polypeptide of the present invention is at least 20, preferably at least 30, more preferably at least 40, still more preferably at least 50, or most preferably at least 100 residues long. The invention also contemplates polypeptides which share at least a 60%, preferably at least a 70%, more preferably at least an 80%, still more preferably a 90%, or most preferably at least a 95% sequence identity over at least 20, preferably at least 30, more preferably at least 40, still more preferably at least 50, or most preferably at least 100 residues with SEQ ID NO:33. SEQ ID NO:33 is a longer novel sequence we have discovered which includes SEQ ID NO:1 as a lesser included sequence (SEQ ID NO:1 is the same as residues 654 to 861 of SEQ ID NO:33).

Such a polypeptide as described above may be (i) one in which one or more of the amino acid residues are substituted (as compared to SEQ ID NO:1) with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethyleneglycol), or (iv) one in which additional amino acids are fused to the mature polypeptide, such as a leader or secretory sequence or a sequence which is employed for purification of the mature polypeptide or a proprotein sequence or mature protein sequence beyond the thrombospondin-repeat domain, or (v) one in which one or more amino acids are deleted from or inserted into the sequence of the polypeptide. Combinations of the above-described types of variations in the peptide sequence are within the scope of the invention. Such polypeptides are deemed to be within the scope of those skilled in the art from the teachings herein.

A polypeptide of the present invention may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains, and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslational natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination.

The polypeptides and polynucleotides of the present invention are preferably provided in an isolated form, and preferably are purified to homogeneity. The term “isolated” means that the material is removed from its original environment (e.g., the natural environment if it is naturally occurring). For example, a naturally occurring polynucleotide or polypeptide present in a living animal is not isolated, but the same polynucleotide or DNA or polypeptide, separated from some or all of the coexisting materials in the natural system, is isolated. Such polynucleotide could be part of a vector and/or such polynucleotide or polypeptide could be part of a composition, and still be isolated in that such vector or composition is not part of its natural environment.

As known in the art “similarity” between two polypeptides is determined by comparing the amino acid sequence and its conserved amino acid substitutes of one polypeptide to the sequence of a second polypeptide. Such conservative substitutions include those described by Dayhoff, The Atlas of Protein Sequence and Structure 5 (1978) and by Argos, EMBO J. 8: 779-785 (1989). For example, amino acids belonging to one of the following groups represent conservative changes:

ala, pro, gly, gin, asn, ser, thr;

cys, ser, tyr, thr;

val, ile, leu, met, ala, phe;

lys, arg, his;

phe, tyr, trp, his; and

asp, glu.

(Note that these grouping are examples; other groupings may represent more relevant choices.)

“Similarity” or “identity” refers to sequence conservation, or “homology”, between two or more peptides or two or more nucleic acid molecules, normally expressed in terms of percentages. When a position in the compared sequences is occupied by the same base or amino acid (“residue”), then the molecules are identical at that position. When a position in two compared peptide sequences is occupied by an amino acid with similar physical properties (a conservative substitution as determined by a given scoring matrix; similarity is thus dependent on the scoring matrix chosen), then the molecules are similar at that position. The percent identity or similarity can be maximized by aligning the compared sequences alongside each other, sliding them back and forth, and conservatively introducing gaps in the sequences where necessary. The percent identity is calculated by counting the number of identical aligning residues dividing by the total length of the aligned region, including gaps in both sequences, and multiplying by 100. Identity would thus be expressed as, e.g., “60% identity over 200 amino acids,” or “57% identity over 250 amino acids.” Similarity is calculated by counting both identities and similarities in the above calculation. For example, the alignment below has 37.5% sequence identity over 56 amino acids ((21 identities/56 residues)×100%), where 56 is the total length of the aligned region.

RTPSDKPVAH--VANPQLQWLNRRANALLANGVE-RDNQLVV--EGLYLIYSQVLF 56 resid.
| | | | || | | | | || | || ||| | | |21 ident.
RAPFKKSWAYLQVAKHKLSW-NK--DGIL-HGVRYQDGNLVIQFPGLYFIICQLQF 56 resid.
First sequence is SEQ ID NO:2; second sequence is SEQ ID NO:3

As a further example, the same alignment below has 55.4% sequence similarity over 56 amino acids ((31 similarities/56 residues)×100% ), where 56 is the total length of the aligned region. In this example, conservative substitutions are indicated by a plus sign and the total similarities is given by the sum of the identities and the conservative substitutions. (As noted above, determination of conservative substitutions is dependent on the scoring matrix chosen. The same alignment below may yield a different value for percent similarity using a different scoring matrix.)

RTPSDKPVAH--VANPQLQWLNRRANALLANGVE-RDNQLVVE--GLYLIYSQVLF 56 resid.
R P K A+ VA +L W N+ + +L +GV +D LV++ GLY I Q+ F 31 simil.
RAPFKKSWAYLQVAKHKLSW-NK--DGIL-HGVRYQDGNLVIQFPGLYFIICQLQF 56 resid.
First sequence is SEQ ID NO:2; second sequence is SEQ ID NO:3

Both of the sequences in the aligned region may be contained within longer, possibliy less homologous sequences. “Unrelated” or “non-homologous” sequences typically share less than 40% identity at the peptide level, preferably less than 25% identity.

The invention further encompasses polynucleotides which code for the above-described polypeptides of the present invention. These polynucleotides may be in the form of RNA or in the form of DNA, which DNA includes cDNA, genomic DNA, and synthetic DNA. The DNA may be double-stranded or single-stranded. The polynucleotides may include: only the coding sequence for the mature polypeptide; the coding sequence for the mature polypeptide and additional coding sequence such as a leader or secretory sequence or a proprotein sequence; the coding sequence for the mature polypeptide (and, optionally, additional coding sequence) and non-coding sequence, such as introns or non-coding -sequence 5′ and/or 3′ of the coding sequence for the mature polypeptide. Thus, the term “polynucleotide encoding a polypeptide” encompasses a polynucleotide which includes only coding sequence for the polypeptide as well as a polynucleotide which includes additional coding and/or non-coding sequence.

The present invention further relates to variants of the herein above-described polynucleotides. The variants of the polynucleotides may be naturally occurring allelic variants of the polynucleotides or non-naturally occurring variants of the polynucleotides. As known in the art, an allelic variant is an alternate form of a polynucleotide sequence which may have a substitution, deletion, or addition of one or more nucleotides which does not substantially alter the function of the encoded polypeptides. Thus, the present invention includes polynucleotides encoding the same mature polypeptide as described in Example 1, below, as well as variants of such polynucleotides which variants include deletion variants, substitution variants, and addition or insertion-variants.

The present invention also includes polynucleotides wherein the coding sequence for the mature polypeptides may be fused to a polynucleotide sequence which aids in expression and secretion of a polypeptide from a host cell, for example, a leader sequence which functions as a secretory sequence for controlling transport of a polypeptide from the cell. The polypeptide having a leader sequence is a preprotein and may have the leader sequence cleaved by the host cell to form the mature form of the polypeptide. The polynucleotides may also encode for a proprotein which is the mature protein plus additional 5′amino acid residues. A mature protein having a prosequence is a proprotein and is an inactive form of the protein. Once the prosequence is cleaved an active mature protein remains. For example, the polynucleotides of the present invention may code for a mature protein or for a protein having a prosequence or for a protein having both a prosequence and a presequence (leader sequence).

The polynucleotides of the present invention may also have the coding sequence fused in frame to a marker sequence which allows for purification of the polypeptide of the present invention. The marker sequence may be, for example, a hexa-histidine tag supplied by a pQE-9 vector to provide for purification of the mature polypeptide fused to the marker in the case of a bacterial host, or, for example, the marker sequence may be a hemagglutinin (HA) tag when a mammalian host, e.g. COS-7 cells, is used. The HA tag corresponds to an epitope derived from the influenza hemagglutinin protein. Wilson et al., 1984, Cell 37: 767. Other tag systems are well-known in the art, including the FLAG tag. The FLAG tag is based on the FLAG marker octapeptide (N-AspTyrLysAspAspAspAspLys-C) (SEQ ID NO:4). The FLAG sequence is hydrophilic and the last 5 amino acids (AspAspAspAspLys) (subsequence of SEQ ID NO:4) represent the target sequence of the protease enterokinase.

The term “gene” means the segment of DNA involved in producing a polypeptide chain; it includes regions preceding and following the coding region (leader and trailer) as well as intervening sequences (introns) between individual coding segments (exons). Fragments of the full length BTL.012 gene may be used as a hybridization probe for a cDNA library to isolate the full length gene and to isolate other genes which have a high sequence similarity to the gene or similar biological activity. Probes of this type typically have at least 20 bases and preferably have at least 30 bases and may contain, for example, 50 or more bases. The probe may also be used to identify a cDNA clone corresponding to a full length transcript and a genomic clone or clones that contain the complete BTL.012 gene including regulatory and promotor regions, exons, and introns. An example of a screen comprises isolating the coding region of the BTL.012 gene by using the known DNA sequence to synthesize an oligonucleotide probe. Labeled oligonucleotides having a sequence complementary to that of the gene of the present invention are used to screen a library of human cDNA, genomic DNA or mRNA to determine which members of the library the pr6be hybridizes to.

The present invention is directed to polynucleotides having at least a 70% identity, preferably at least 80% identity, more preferably at least a 90% identity, still more preferably at least a 95% identity, and most preferably at least 98% identity to a polynucleotide which encodes a polypeptide of the present invention, as well as fragments thereof, which fragments have at least 20 bases and preferably have at least 30 bases and more preferably have at least 50 bases, and to polypeptides encoded by such polynucleotides. One embodiment of the present invention is given by SEQ ID NO:34.

The present invention also relates to vectors that include polynucleotides of the present invention as above described, host cells that are genetically engineered with vectors of the invention, and the production of polypeptides of the invention by recombinant techniques. Host cells may be genetically engineered (transduced or transformed or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the BTL.012 genes. The culture conditions, such as temperature, pH and the like, are those previously used with the host cell selected for expression, and will be apparent to the ordinarily skilled artisan. The polynucleotide of the present invention may be employed for producing a polypeptide by recombinant techniques.

Thus, for example, the polynucleotide sequence may be included in any one of a variety of expression vehicles, in particular vectors or plasmids for expressing a polypeptide. Such vectors include chromosomal, non-chromosomal and synthetic DNA sequences, e.g., derivatives of SV40; bacterial plasmids; phage DNA; yeast plasmids; vectors derived from combinations of plasmids and phage DNA, viral DNA such as vaccinia, adenovirus, fowl pox virus, and pseudorabies. However, any other vector or plasmid may be used as long as they are replicable and viable in the host.

The appropriate DNA sequence may be inserted into the vector by a variety of procedures. Such procedures and others are deemed to be within the scope of those skilled in the art. The DNA sequence in the expression vector is operatively linked to an appropriate expression control sequence(s) (promoter) to direct mRNA synthesis. As representative examples of such promoters, there may be mentioned: LTR or SV40 promoter, the E. coil. lac or trp, the phage lambda PL promoter and other promoters known to control expression of genes in prokaryotic or eukaryotic cells or their viruses. The expression vector may also contain a ribosome binding site for translation initiation and a transcription terminator. The vector may also include appropriate sequences for amplifying expression. In addition, the expression vectors preferably contain a gene to provide a phenotypic trait for selection of transformed host cells such as dihydrofolate reductase or neomycin resistance for eukaryotic cell culture, or such as tetracycline or ampicillin resistance in E. coli. The vector containing the appropriate DNA sequence as herein above described, as well as an appropriate promoter or control sequence, may be employed to transform an appropriate host to permit the host to express the protein. As representative examples of appropriate hosts, there may be mentioned: bacterial cells, such as E. coli, Salmonella typhimurium, Streptomyces; fungal cells, such as yeast; insect cells, such as Drosophila S2 and Spodoptera Sf9; animal cells such as CHO, COS or Bowes melanoma; plant cells, etc. The selection of an appropriate host is deemed to be within the scope of those skilled in the art from the teachings herein.

The present invention also includes recombinant constructs comprising one or more of the sequences as broadly described above. The constructs comprise a vector, such as a plasmid or viral vector, into which a sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art and are commercially available. The following vectors are provided by way of example. Bacterial: pQE70, pQE60, pQE-9 (Qiagen), pBS, phagescript, psiX174, pBluescript SK, pBsKS, pNH8a, pNH16a, pNH18a, pNH46a (Stratagene), pTRC99A, pKK223-3, pKK233-3, pDR540, PRIT5 (Pharmacia). Eukaryotic: pWLneo, pSV2cat, pOG44, pXT1, pSG (Stratagene) pSVK3, pBPV, pMSG, PSVL (Pharmacia). However, any other plasmid or vector may be used as long as they are viable or can be made viable in the host. Promoter regions can be selected from any desired gene using CAT (chloramphenicol acetyl transferase) vectors or other vectors with selectable markers. Two appropriate vectors are pKK232-8 and pCM7. Particular named bacterial promoters include laci, lacZ, T3, T7, gpt, lambda PR, PL and trp. Eukarvotic promoters include CMV immediate early, HSV thymidine kinase, early and late SV40, LTRs from retrovirus, and mouse metallothionein-I. Selection of the appropriate vector and promoter is well within the level of ordinary skill in the art.

The present invention also relates to host cells containing the above-described construct. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation. The constructs in host cells can be used in a conventional manner to produce the gene product encoded by the recombinant sequence. Alternatively, the polypeptides of the invention can be synthetically produced by conventional peptide synthesizers.

Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook et al. (Molecular Cloning: A Laboratory Manual, Second Edition, Cold Spring Harbor, N.Y., 1989; the disclosure of which is hereby incorporated by reference).

Transcription of a DNA encoding the polypeptides of the present invention by higher eukaryotes is increased by inserting an enhancer sequence into the vector. Enhancers are cis-acting elements of DNA, usually from about 10 to 300 bp, that act on a promoter to increase its transcription. Examples include the SV40 enhancer on the late side of the replication origin (bp 100 to 270), a cytomegalovirus early promoter enhancer, a polyoma enhancer on the late side of the replication origin, and adenovirus enhancers. Generally, recombinant expression vectors will include origins of replication and selectable markers permitting transformation of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), alpha factor, acid phosphatase, or heat shock proteins, among others. The heterologous structural sequence is assembled in appropriate phase with translation, initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

Useful expression vectors for bacterial use are constructed by inserting a structural DNA sequence encoding a desired protein together with suitable translation, initiation, and termination signals in operable reading phase with a functional promoter. The vector will comprise one or more phenotypic selectable markers and an origin of replication to ensure maintenance of the vector and, if desirable, to provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice. Useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEM1 (Promega Biotec, Madison, Wis.) These pBR322 “backbone” sections are combined with an appropriate promoter and the structural sequence to be expressed.

After transformation of a suitable host strain and growth of the host strain to an appropriate cell density, the selected promoter may be de-repressed, if necessary, by appropriate means (e.g., temperature shift or chemical induction) and the cells may be cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents.

Various mammalian cell culture systems can also be employed to express recombinant protein. Examples of mammalian expression systems include the COS-7 lines of monkey kidney fibroblasts (82) and other cell lines capable of expressing protein from a compatible vector, for example, the C127, 3T3, CHO, HeLa and BHK cell lines. Mammalian expression vectors will generally comprise an origin of replication, a suitable promoter and enhancer, and also any necessary ribosome binding sites, polyadenylation site, splice donor and acceptor sites, transcription termination sequences, and 5′ flanking nontranscribed sequences. DNA sequences derived from the SV40 viral genome, for example, SV40 origin, early promoter, enhancer, splice, and polyadenylation sites may be used to provide the required non-transcribed genetic elements.

The polypeptide of the present invention may be recovered and purified from recombinant cell cultures by methods used heretofore, including ammonium sulfate or ethanol precipitation, acid extraction, anion or cation exchange chromatography, phosphocellulose chromatography, hydrophobic interaction chromatography, affinity chromatography, hydroxyapatite chromatography and lectin chromatography. Protein refolding steps can be used, as necessary, in completing configuration of the mature protein. Finally, high performance liquid chromatography (HPLC) can be employed for final purification steps.

The polypeptide of the present invention may be a naturally purified product, or a product of chemical synthetic-procedures, or produced by recombinant techniques from a prokaryotic or eukaryotic host (for example, by bacterial, yeast, higher plant, insect and mammalian cells in culture). Depending upon the host employed in a recombinant production procedure, the polypeptides of the present invention may be glycosylated with mammalian or other eukaryotic carbohydrates or may be non-glycosylated. Polypeptides of the present invention may also include an initial methionine amino acid residue.

Polypeptides of the present invention, or polynucleotides coding for polypeptides of the present invention, may be used in a process of gene therapy. Such gene therapy may be involved in the treatment of a disease or clinical condition which may include but not limited to cancer, wound healing, diabetic retinopathies, macular degeneration, and cardiovascular diseases. Fr example, cells may be engineered with a polynucleotide (DNA or RNA) encoding for the polypeptide ex vivo, the engineered cells are then provided to a patient to be treated with the polypeptide. Such methods are well-known in the art. For example, cells may be engineered by procedures known in the art by use of a retroviral particle containing RNA encoding for the polypeptide of the present invention.

Both in vitro and in vivo gene therapy methodologies are contemplated. Several methods for transferring potentially therapeutic genes to defined cell populations are known. See, e.g., Mulligan (1993) Science 260: 926-31. These methods include:

1) Direct gene transfer. See, e.g., Wolff et al (1990) Science 247:1465-68.

2) Liposome-mediated DNA transfer. See, e.g., Caplen at al. (1995) Nature Med. 3: 39-46; Crystal (1995) Nature Med. 1:15-17; Gao and Huang (1991) Biochem. Biophys. Res. Comm. 179:280-85.

3) Retrovirus-mediated DNA transfer. See, e.g., Kay et al. (1993) Science, 262:117-19; Anderson (1992) Science 256:808-13. Retroviruses from which the retroviral plasmid vectors hereinabove mentioned may be derived include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumor virus. In one embodiment, the retroviral plasmid vector is derived from Moloney Murine Leukemia Virus.

4) DNA Virus-mediated DNA transfer. Such DNA viruses include adenoviruses (preferably Ad-2 or Ad-5 based vectors), herpes viruses (preferably herpes simplex virus based vectors), and parvoviruses (preferably “defective” or non-autonomous parvovirus based vectors, more preferably adeno-associated virus based vectors, most preferably AAV-2 based vectors). See, e.g., Ali et al. (1994) Gene Therapy, 1:367-84; U.S. Pat. No. 4,797,368, incorporated herein by reference, and U.S. Pat. No. 5,139,941, incorporated herein by reference. Adenoviruses have the advantage that they have a broad host range, can infect quiescent or terminally differentiated cells, such as neurons or hepatocytes, and appear essentially non-oncogenic. Adenoviruses do not appear to integrate into the host genome. Because they exist extrachromosomally, the risk of insertional mutagenesis is greatly reduced. Adeno-associated viruses exhibit similar advantages as adenoviral-based vectors. However, AAVs exhibit site-specific integration on human chromosome 19.

The choice of a particular vector system for transferring the gene of interest will depend on a variety of factors. One important factor is the nature of the target cell population. Although retroviral vectors have been extensively studied and used in a number of gene therapy applications, these vectors are generally unsuited for infecting non-dividing cells. In addition, retroviruses have the potential for oncogenicity. However, recent developments in the field of lentiviral vectors may circumvent some of these limitations. See Naldini et al. (1996) Science 272:263-7.

According to this embodiment, gene therapy with DNA encoding a polypeptide of the present invention is provided to a patient in need thereof, concurrent with, or immediately after diagnosis. The skilled artisan will appreciate that any suitable gene therapy vector containing DNA encoding a polypeptide of the present invention may be used in accordance with this embodiment. The techniques for constructing such a vector are known. See, e.g., Anderson (1998) Nature, 392 25-30; Verma (1998) Nature, 389 239-42. Introduction of the vector to the target site may be accomplished using known techniques.

The present invention also relates to a diagnostic assay for detecting levels of polypeptides of the present invention, e.g. in various tissues, since an over-expression of the proteins compared to normal control tissue samples may detect the presence of abnormal cellular proliferation, for example, a tumor. Assays used to detect levels of protein in a sample derived from a host are well-known to those of skill in the art and include radioimmunoassays, competitive-binding assays, Western Blot analysis, ELISA assays and “sandwich” type assays.

The polypeptides of the present invention can be used as an immunogen to produce antibodies thereto. These antibodies can be, for example, polyclonal or monoclonal antibodies. The present invention also includes chimeric, single chain, and humanized antibodies, as well as Fab fragments, or the product of an Fab expression library. Various procedures known in the art may be used for the production of such antibodies and fragments.

Antibodies generated against the polypeptides of the present invention can be obtained by direct injection of the polypeptides into an animal or by administering the polypeptides to an animal, preferably a nonhuman. The antibody so obtained will then bind the polypeptides itself In this manner, even a sequence encoding only a fragment of the polypeptides can be used to generate antibodies binding the whole native polypeptides. Such antibodies can then be used to isolate the polypeptide from tissue expressing that polypeptide or as a diagnostic reagent.

For preparation of monoclonal antibodies, any technique which provides antibodies produced by continuous cell line cultures can be used. See generally Antibodies: A Laboratory Manual, Harlow and Lane, eds. (1988) Cold Spring Harbor Laboratory. Examples include the hybridoma technique (Kohler and Milstein (1975) Nature 256:495-97), the trioma technique, the human B-cell hybridoma technique (Kozbor et al. (1983) Immunology Today, 4:72), and the EBV-hybridoma technique to produce human monoclonal antibodies (Cole et al. (1985) in Monoclonal Antibodies and Cancer Therapy, Alan R. Liss, Inc., pp. 77-96).

Techniques described for the production of single chain antibodies (U.S. Pat. No. 4,946,778) can be adapted to produce single chain antibodies to immunogenic polypeptide products of this invention. Also, transgenic mice may be used to express humanized antibodies to immunogenic polypeptide products of this invention. Humanized antibodies may also be produced by methods described in U.S. Pat. Nos. 5,530,101; 5,585,089; 5,693,761; and 5,693,762, incorporated herein by reference.

### Identification of Novel Protein and Comparison with Known Sequences

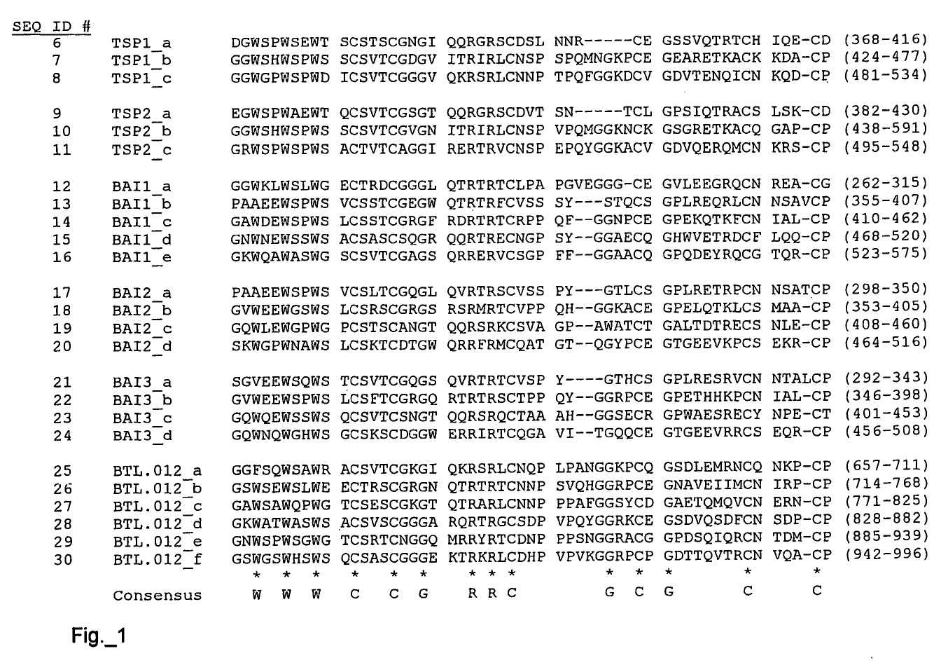

Random 5′ and 3′ sequences were obtained from a cDNA library of clones constructed from poly-A+ RNA prepared from mesenchymal stem cells (MSC) treated with dexamethasone. All these sequences were searched against the Genbank, Genpept, mm_uni_all, rn_uni_all, gbest, and hs_uni_fl databases using Blastn and Blastx (Altschul et al., Basic Local Alignment Search Tool, J. Mol. Biol. (1990) 215: 403-10). These database files are publicly available at the NCBI on-line database at http://www.ncbi.nih.nlm.gov (National Cener for Biotechnology Information, Bethesada, Md.). The _uni_database files refer to the Unigene files at NCBI for mm (mouse) rn (rat) and hs (human). One sequence, denoted M2DEX19_A5.T7X, was discovered to be homologous to a predicted Ig superfamily repeat (I-type). The M2DEX19_A5.T7X sequence was then used to screen a human fetal tissue cDNA libarary. One positive clone was identified from this library and sequenced using the ABI dye terminator method. The final sequence was aligned using the Sequencher program (Gene Codes Corp., Ann Arbor, Mich.). The resulting sequence contained a 2.4 kb segment with an open reading frame encoding a peptide of 796 amino acids. The amino acid sequence was analyzed by Profile scan algorithm (Swiss Institute for Experimental Cancer Research, Lausanne, Switzerland; software by Phillip Bucher, available at: http://www.isrec.isb-sib.ch/sib-isrec/pftools). Six thrombospondin type I repeats are located at the C-terminus of this peptide (FIG. 1).

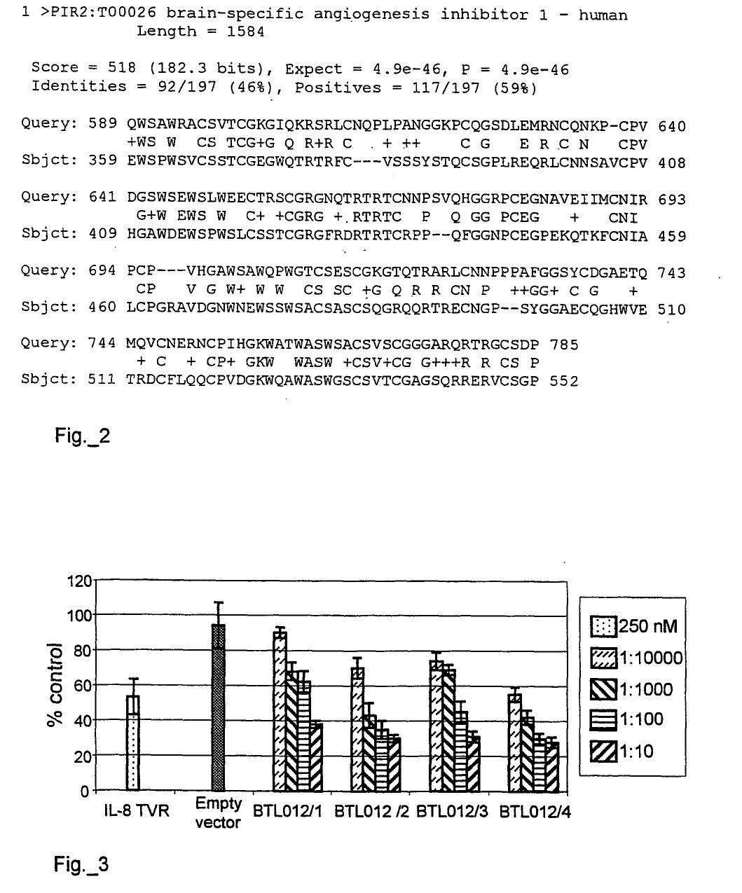

A BLASTX search of this thrombospondin repeat domain showed that it shares 59% homology and 46% identity with human brain-specific angiogenesis inhibitor I (see FIG. 2). A recent search against available public databases, including Genbank and Genpept, confirmed that this sequence was a novel sequence.

A portion (sequence given by SEQ ID NO:1) of the thrombospondin repeat domain was cloned into pFLAG-CMV-1 vector. The pFLAG-CMV-1 vector is a transient expression vector for expression and secretion of N-terminal FLAG fusion proteins in mammalian cells. The preprotrypsin leader sequence precedes the FLAG sequence. Transcription of FLAG-fusion constructs is driven by the promoter-regulatory region of the human cytomegalovirus. A fusion protein, FLAG-BTL012, was expressed with a preprotrypsin signal peptide at its N-terminus followed by a FLAG tag. The signal peptide and the FLAG tag are not expected to adversely interact with the remaining protein structure, thus allowing the fusion protein to be an effective model for the BTL.012 protein which lacks the signal peptide/FLAG tag and other BTL.012-like proteins, including the protein described by SEQ ID NO:33. The pFLAG-CMV-1 vector containing the BTL.012 coding sequence was then transfected into HEK293EBNA cells (Invitrogen, Carlsbad, Calif.). Three days after transfection, the supernatant and the pellet were collected. Western blot analysis of the supernatant and pellet revealed that this fusion protein was expressed in HEK293EBNA cells and secreted into the conditioned medium.

### EXAMPLE 1

The effect of BTL.012 protein supernatants was evaluated in vitro using the HUVEC MATRIGEL assay. This assay mimics endothelial cell capillary organization and is a standard in vitro assay used to evaluate angiogenic mechanisms.

Conditioned medium was collected from HEK293EBNA cells producing the FLAG-BTLO12 fusion protein as above. The conditioned medium was centrifuged to remove cell debris and the supernatants were recovered. The supernatants were added at different concentrations to HUVEC grown in culture on MATRIGEL: Twenty-four hours later cells were fixed and evaluated for capillary-like organization. Measurement of the capillary-like structures in each well allows a quantitative analysis of the biological effect of tested compounds.

The BTL.0 1 2-containing supernatants significantly inhibited HUVEC capillary-like organization. Results from a series of representative experiments are presented in FIG. 3. In this experiment supernatants containing BTL.012 protein were added to the cells at various dilutions from 1:10 to 1:10,000 with log increment. Four different clones labeled BTL012/1, BTL012/2, BTL012/3 and BTL012/4 were used to generate BTL.012-protein-containing supernatants. The results show that the supernatants inhibited capillary-like organization in a dose dependent manner (see FIG. 3). In this experiment, IL-8-TVR, an IL-8 mutein that has been shown to have an inhibitory effect in this assay, was added at a concentration of 250 nM. As a negative control, protein supernatant from cells that have been transfected with an empty vector containing the CMV-1 promoter alone (pFLAG-CMV-1) was added to the cells at the highest dilution used with the test protein supernatants. This control did not have any significant effect on HUVEC capillary-like organization.

### EXAMPLE 2

The effect of BTL.012 purified protein was evaluated in vitro using the HUVEC and MLuEC cells in the MATRIGEL assay. Supernatants were collected from HEK293EBNA cells producing the FLAG-BTL012 fusion protein, as above. The supernatants were filtered through a 0.22 um filter before the purification. The filtered supernatant was then added to an anti-FLAG M2 affinity column prepared by covalently attaching the purified murine IgG1 M2 monoclonal antibody to agarose beads. The column was washed three times with TBS (50 mM Tris-HCl, 150 mM NaCl, pH 7.4), and BTL.012 was then eluted with 100 ug/ml FLAG peptide. The BTL.012 containing elute was then dialysed to eliminate the small FLAG peptide contamination. Test samples of the protein were added at different concentrations to HUVEC or MLuEC grown in culture on MATRIGEL. Twenty-four hours later cells were fixed and evaluated for capillary-like organization.

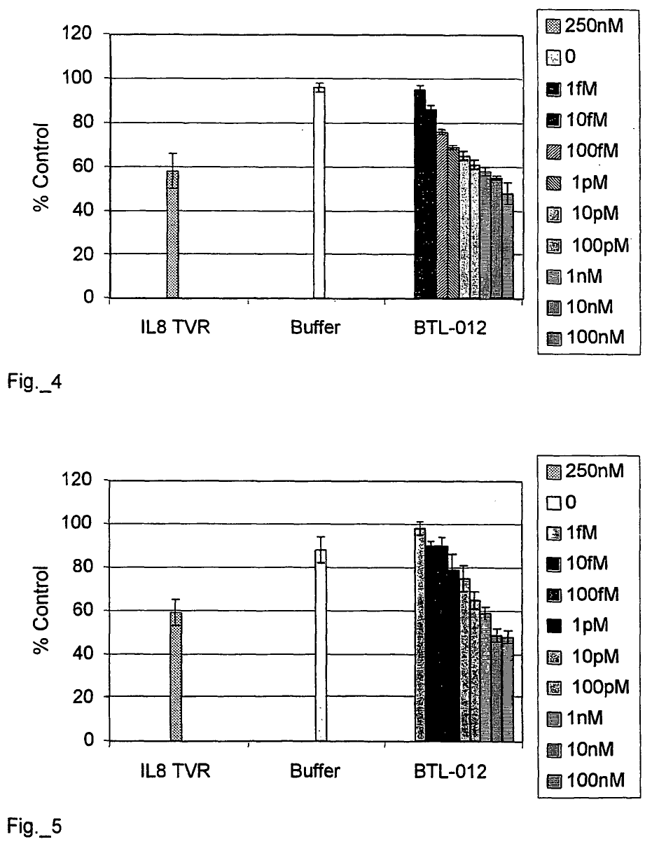

The BTL.012 protein significantly inhibited HUVEC or MLuEC capillary-like organization. Results are presented in FIGS. 4 and 5. In these experiments BTL.012 protein was added to the cells at various concentrations from 100 nM to 1 fM with log increment. The results show that the protein inhibited capillary-like organization in a dose dependent manner (see FIGS. 4 and 5). In these experiments, IL-8-TVR, an IL-8 mutein that has been shown to have an inhibitory effect in this assay, was added at a concentration of 250 nM. As a negative control, the diluent (D-PBS) was added to the cells at the highest volume used with the test protein. This control did not have any significant effect on the capillary-like organization.

### Conclusion

This invention may be relevant to any disease where angiogenesis is involved, including but not limited to cancer, wound healing, diabetic retinopathies, macular degeneration, and cardiovascular diseases. In addition to their potential therapeutic use, the polypeptides of the present invention may find use in diagnostic applications, as may the polynucleotides which code for the polypeptides of the present invention, and as may antibodies to the polypeptides of the present invention.

The above examples are intended to illustrate the invention and it is thought variations will occur to those skilled in the art. Accordingly, it is intended that the scope of the invention should be limited only by the claims below.

What is claimed is:

1. A method of modulating angiogenesis at a site, the method comprising causing an effective amount of a composition comprising a BTL.012-like protein to be supplied to the site.

2. The method of claim 1 wherein the BTL.012-like protein has an amino acid sequence identical to SEQ ID NO:1.

3. The method of claim 1 wherein the BTL.012-like protein has an amino acid sequence which is at least 60% identical over at least 40 residues to SEQ ID NO:1.

4. The method of claim 1 wherein the BTL.0 12-like protein has an amino acid sequence which is at least 70% identical over at least 30 residues to SEQ ID NO:1.

5. A method of modulating the formation of cells into capillary-like structures comprising contacting the cells with a biologically effective amount of a composition comprising a BTL.012-like protein.

6. The method of claim 5 wherein the cells are endothelial cells of human origin.

7. A protein characterized by having a deduced amino acid sequence which is at least 60% identical over 40 residues to SEQ ID NO:1.

8. The protein according to claim 7, wherein the deduced amino acid sequence is at least 80% identical over 50 residues to SEQ ID NO:1.

9. A pharmaceutical composition for modulating angiogenesis comprising a protein characterized by having a deduced amino acid sequence which is at least 60% identical over 40 residues to SEQ ID NO:1 and a pharmaceutically acceptable carrier.

10. The method of claim 1, wherein the site is within a human patient and the protein is supplied to the site via a pharmaceutical composition according to claim 9.

11. The method of claim 10, wherein the site is within a human patient and the protein is supplied to the site via a process of gene therapy.

12. A method for preventing, treating, or ameliorating a medical condition in an individual, the method comprising providing a source of an effective amount of at least one protein according to claim 7 to the individual.

13. The method of claim 12, wherein the protein is supplied to the individual by providing to the individual a source of a polynucleotide encoding the protein and expressing the protein in vivo.

14. The method of claim 12, wherein the medical condition is selected from the group consisting of cancer, metastasis, diabetic retinopathy, macular degeneration, cardiovascular disease, and a wound.

15. A polynucleotide selected from the group consisting of (a) a polynucleotide coding for a protein according to claim 7; (b) a polynucleotide complementary to (a); (c) a polynucleotide having at least 90% identity over at least 20 bases to SEQ ID NO:34; and (d) a polynucleotide complementary to (c).

16. The polynucleotide according to claim 15, wherein the polynucleotide is operably linked within an expression vector to a promoter, the expression vector thus being capable of being used to express the protein according to claim 1.

17. A method for producing a protein according to claim 7 comprising the steps of
(a) introducing an expression vector capable of expressing the protein according to claim 7 into a cell capable of expressing the protein according to claim 7,
(b) growing cells resulting from step (a) under conditions sufficient to allow the cells to express the protein according to claim 7, and
(c) recovering the protein according to claim 7 from the result of step (b).

18. An antibody against a protein according to claim 7.

19. A method for diagnosing a disease or medical condition or susceptibility to a disease or medical condition, the disease or medical condition related to inadequate or excess expression of a protein according to claim 7, the method comprising the steps
(a) determining the level of expression of said protein in a sample; and
(b) comparing the level of expression of said protein against a standard to make a diagnosis.

20. The method of claim 19, wherein the medical condition is selected from the group consisting of cancer, metastasis, diabetic retinopathy, macular degeneration, cardiovascular disease, and a wound.

21. A protein characterized by having a deduced amino acid sequence which is at least 60% identical over 40 residues to SEQ ID NO:33.


SEQUENCE LISTING (34 sequences)

SEQ ID NO: 1 - protein, 208 aa, Homo sapiens
QVHGGFSQWSAWRACSVTCGKGIQKRSRLCNQPLPANGGKPCQGSDLEMRNCQNKPCPVDGSWSEWSLWEECTRSCGRGNQTRTRTCNNPSVQHGGRPCEGNAVEIIMCNIRPCPVHGAWSAWQPWGTCSESCGKGTQTRARLCNNPPPAFGGSYCDGAETQMQVCNERNCPIHGKWATWASWSACSVSCGGGARQRTRGCSDPVPQY

SEQ ID NO: 2 - protein, 51 aa, Artificial Sequence
  Random sequence
RTPSDKPVAHVANPQLQWLNRRANALLANGVERDNQLVVEGLYLIYSQVLF

SEQ ID NO: 3 - protein, 52 aa, Artificial Sequence
  Random sequence
RAPFKKSWAYLQVAKHKLSWNKDGILHGVRYQDGNLVIQFPGLYFIICQLQF

SEQ ID NO: 4 - protein, 8 aa, Artificial Sequence
  FLAG sequence for expressed protein
DYKDDDDK

SEQ ID NO: 5 - protein, 6 aa, Artificial Sequence
  Sequence with antineoangiogenic activity
CSVTCG

SEQ ID NO: 6 - protein, 50 aa, Artificial Sequence
  Isolated type 1 thrombospondin domain sequence
DGWSPWSEWTSCSTSCGNGIQQRGRSCDSLNNRCEGSSVQTRTCHIQECD

SEQ ID NO: 7 - protein, 55 aa, Artificial Sequence
  Isolated type 1 thrombospondin domain sequence
GGWSHWSPWSSCSVTCGDGVITRIRLCNSPSPQMNGKPCEGEARETKACKKDACP

SEQ ID NO: 8 - protein, 55 aa, Artificial Sequence
  Isolated type 1 thrombospondin domain sequence
GGWGPWSPWDICSVTCGGGVQKRSRLCNNPTPQFGGKDCVGDVTENQICNKQDCP

SEQ ID NO: 9 - protein, 50 aa, Artificial Sequence
  Isolated type 1 thrombospondin domain sequence
EGWSPWAEWTQCSVTCGSGTQQRGRSCDVTSNTCLGPSIQTRACSLSKCD

SEQ ID NO: 10 - protein, 55 aa, Artificial Sequence
  Isolated type 1 thrombospondin domain sequence
GGWSHWSPWSSCSVTCGVGNITRIRLCNSPVPQMGGKNCKGSGRETKACQGAPCP

SEQ ID NO: 11 - protein, 55 aa, Artificial Sequence
  Isolated type 1 thrombospondin sequence
GRWSPWSPWSACTVTCAGGIRERTRVCNSPEPQYGGKACVGDVQERQMCNKRSCP

SEQ ID NO: 12 - protein, 54 aa, Artificial Sequence
  Isolated type 1 thrombospondin domain sequence
GGWKLWSLWGECTRDCGGGLQTRTRTCLPAPGVEGGGCEGVLEEGRQCNREACG

SEQ ID NO: 13 - protein, 53 aa, Artificial Sequence
  Isolated type 1 thrombospondin domain sequence
PAAEEWSPWSVCSSTCGEGWQTRTRFCVSSSYSTQCSGPLREQRLCNNSAVCP

SEQ ID NO: 14 - protein, 53 aa, Artificial Sequence
  Isolated type 1 thrombospondin domain sequence
GAWDEWSPWSLCSSTCGRGFRDRTRTCRPPQFGGNPCEGPEKQTKFCNIALCP

SEQ ID NO: 15 - protein, 53 aa, Artificial Sequence
  Isolated type 1 thrombospondin domain sequence
GNWNEWSSWSACSASCSQGRQQRTRECNGPSYGGAECQGHWVETRDCFLQQCP

SEQ ID NO: 16 - protein, 53 aa, Artificial Sequence
  Isolated type 1 thrombospondin domain sequence
GKWQAWASWGSCSVTCGAGSQRRERVCSGPFFGGAACQGPQDEYRQCGTQRCP

SEQ ID NO: 17 - protein, 53 aa, Artificial Sequence
  Isolated type 1 thrombospondin domain sequence
PAAEEWSPWSVCSLTCGQGLQVRTRSCVSSPYGTLCSGPLRETRPCNNSATCP

SEQ ID NO: 18 - protein, 53 aa, Artificial Sequence
  Isolated type 1 thrombospondin domain sequence
GVWEEWGSWSLCSRSCGRGSRSRMRTCVPPQHGGKACEGPELQTKLCSMAACP

SEQ ID NO: 19 - protein, 53 aa, Artificial Sequence
  Isolated type 1 thrombospondin domain sequence
GQWLEWGPWGPCSTSCANGTQQRSRKCSVAGPAWATCTGALTDTRECSNLECP

SEQ ID NO: 20 - protein, 53 aa, Artificial Sequence
  Isolated type 1 thrombospondin domain sequence
SKWGPWNAWSLCSKTCDTGWQRRFRMCQATGTQGYPCEGTGEEVKPCSEKRCP

SEQ ID NO: 21 - protein, 52 aa, Artificial Sequence
  Isolated type 1 thrombospondin domain sequence
SGVEEWSQWSTCSVTCGQGSQVRTRTCVSPYGTHCSGPLRESRVCNNTALCP

SEQ ID NO: 22 - protein, 53 aa, Artificial Sequence
  Isolated type 1 thrombospondin domain sequence
GVWEEWSPWSLCSFTCGRGQRTRTRSCTPPQYGGRPCEGPETHHKPCNIALCP

SEQ ID NO: 23 - protein, 53 aa, Artificial Sequence
  Isolated type 1 thrombospondin domain sequence
GQWQEWSSWSQCSVTCSNGTQQRSRQCTAAAHGGSECRGPWAESRECYNPECT

SEQ ID NO: 24 - protein, 53 aa, Artificial Sequence
  Isolated type 1 thrombospondin domain sequence
GQWNQWGHWSGCSKSCDGGWERRIRTCQGAVITGQQCEGTGEEVRRCSEQRCP

SEQ ID NO: 25 - protein, 55 aa, Artificial Sequence
  Isolated type 1 thrombospondin domain sequence
GGFSQWSAWRACSVTCGKGIQKRSRLCNQPLPANGGKPCQGSDLEMRNCQNKPCP

SEQ ID NO: 26 - protein, 55 aa, Artificial Sequence
  Isolated type 1 thrombospondin domain sequence
GSWSEWSLWEECTRSCGRGNQTRTRTCNNPSVQHGGRPCEGNAVEIIMCNIRPCP

SEQ ID NO: 27 - protein, 55 aa, Artificial Sequence
  Isolated type 1 thrombospondin domain sequence
GAWSAWQPWGTCSESCGKGTQTRARLCNNPPPAFGGSYCDGAETQMQVCNERNCP

SEQ ID NO: 28 - protein, 55 aa, Artificial Sequence
  Isolated type 1 thrombospondin domain sequence
GKWATWASWSACSVSCGGGARQRTRGCSDPVPQYGGRKCEGSDVQSDFCNSDPCP

SEQ ID NO: 29 - protein, 55 aa, Artificial Sequence
  Isolated type 1 thrombospondin domain sequence
GNWSPWSGWGTCSRTCNGGQMRRYRTCDNPPPSNGGRACGGPDSQIQRCNTDMCP

SEQ ID NO: 30 - protein, 55 aa, Artificial Sequence
  Isolated type 1 thrombospondin domain sequence
GSWGSWHSWSQCSASCGGGEKTRKRLCDHPVPVKGGRPCPGDTTQVTRCNVQACP

SEQ ID NO: 31 - protein, 197 aa, Homo sapiens
QWSAWRACSVTCGKGIQKRSRLCNQPLPANGGKPCQGSDLEMRNCQNKPCPVDGSWSEWSLWEECTRSCGRGNQTRTRTCNNPSVQHGGRPCEGNAVEIIMCNIRPCPVHGAWSAWQPWGTCSESCGKGTQTRARLCNNPPPAFGGSYCDGAETQMQVCNERNCPIHGKWATWASWSACSVSCGGGARQRTRGCSDP

SEQ ID NO: 32 - protein, 194 aa, Homo sapiens
EWSPWSVCSSTCGEGWQTRTRFCVSSSYSTQCSGPLREQRLCNNSAVCPVHGAWDEWSPWSLCSSTCGRGFRDRTRTCRPPQFGGNPCEGPEKQTKFCNIALCPGRAVDGNWNEWSSWSACSASCSQGRQQRTRECNGPSYGGAECQGHWVETRDCFLQQCPVDGKWQAWASWGSCSVTCGAGSQRRERVCSGP

SEQ ID NO: 33 - protein, 1336 aa, Homo sapiens
TPIGRPRIRHQDKRTVDLTVQVPPSIADEPTDFLVTKHAPAVITCTASGVPFPSIHWTKNGIRLLPRGDGYRILSSGAIEILATQLNHAGRYTCVARNAAGSAHRHVTLHVHEPPVIQPQPSELHVILNNPILLPCEATGTPSPFITWQKEGINVNTSGRNHAVLPSGGLQISRAVREDAGTYMCVAQNPAGTALGKIKLNVQVPPVISPHLKEYVIAVDKPITLSCEADGLPPPDITWHKDGRAIVESIRQRVLSSGSLQIAFVQPGDAGHYTCMAANVAGSSSTSTKLTVHVPPRIRSTKGHYTVNENSQAILPCVADGIPTPAINWKKDNVLLANLLGKYTAEPYGELILENVVLEDSGFYTCVANNAAGEDTHTVSLTVHVLPTFTELPGDVSLNKGEQLRLSCKATGIPLPKLTWTFNNNIIPAHFDSVNGHSELVIERVSKEDSGTYVCTAENSVGFVKAIGFVYVKEPPVFKGDYPSNWIEPLGGNAILNCEVKGDPTPTIQWNRKGVDIEISHRIRQLGNGSLAIYGTVNEDAGDYTCVATNEAGVVERSMSLTLRSPPIITLEPVETVINAGGKIILNCQATGEPQPTITWSRQGHSISWDDRVNVLSNNSLYIADAQKEDTSEFECVARNLMGSVLVRVPVIVQVHGGFSQWSAWRACSVTCGKGIQKRSRLCNQPLPANGGKPCQGSDLEMRNCQNKPCPVDGSWSEWSLWEECTRSCGRGNQTRTRTCNNPSVQHGGRPCEGNAVEIIMCNIRPCPVHGAWSAWQPWGTCSESCGKGTQTRARLCNNPPPAFGGSYCDGAETQMQVCNERNCPIHGKWATWASWSACSVSCGGGARQRTRGCSDPVPQYGGRKCEGSDVQSDFCNSDPCPTHGNWSPWSGWGTCSRTCNGGQMRRYRTCDNPPPSNGGRACGGPDSQIQRCNTDMCPVDGSWGSWHSWSQCSASCGGGEKTRKRLCDHPVPVKGGRPCPGDTTQVTRCNVQACPGGPQRARGSVIGNINDVEFGIAFLNATITDSPNSDTRIIRAKITNVPRSLGSAMRKIVSILNPIYWTTAKEIGEAVNGFTLTNAVFKRETQVEFATGEILQMSHIARGLDSDGSLLLDIVVSGYVLQLQSPAEVTVKDYTEDYIQTGPGQLYAYSTRLFTIDGISIPYTWNHTVFYDQAQGRMPFLVETLHASSVESDYNQIEETLGFKIHASISKGDRSNQCPSGFTLDSVGPFCADEDECAAGNPCSHSCHNAMGTYYCSCPKGLTIAADGRTCQDIDECALGRHTCHAGQDCDNTIGSYRCVVRCGSGFRRTSDGLSCQDINECQESSPVTSAVSMP

SEQ ID NO: 34 - DNA, 4073 bp, Homo sapiens
actcctatagggcggccgcgaattcggcaccaggataaaagaactgtggatctcactgtccaagttccaccttccatagctgatgagcctacagatttcctagtaaccaaacatgccccagcagtaattacctgcactgcttcgggagttccatttccctcaattcactggaccaaaaatggtataagactgcttcccaggggagatggctatagaattctgtcctcaggagcaattgaaatacttgccacccaattaaaccatgctggaagatacacttgtgtcgctaggaatgcggctggctctgcacatcgacacgtgacccttcatgttcatgagcctccagtcattcagccccaaccaagtgaactacacgtcattctgaacaatcctattttattaccatgtgaagcaacagggacacccagtcctttcattacttggcaaaaagaaggcatcaatgttaacacttcaggcagaaaccatgcagttcttcctagtggcggcttacagatctccagagctgtccgagaggatgctggcacttacatgtgtgtggcccagaacccggctggtacagccttgggcaaaatcaagttaaatgtccaagttcctccagtcattagccctcatctaaaggaatatgttattgctgtggacaagcccatcacgttatcctgtgaagcagatggcctccctccgcctgacattacatggcataaagatgggcgtgcaattgtggaatctatccgccagcgcgtcctcagctctggctctctgcaaatagcatttgtccagcctggtgatgctggccattacacgtgcatggcagccaatgtagcaggatcaagcagcacaagcaccaagctcaccgtccatgtaccacccaggatcagaagtacaaaaggacactacacggtcaatgagaattcacaagccattcttccatgcgtagctgatggaatccccacaccagcaattaactggaaaaaagacaatgttcttttagctaacttgttaggaaaatacactgctgaaccatatggagaactcattttagaaaatgttgtgctggaggattctggcttctatacctgtgttgctaacaatgctgcaggtgaagatacacacactgtcagcctgactgtgcatgttctccccacttttactgaacttcctggagacgtgtcattaaataaaggagaacagctacgattaagctgtaaagctactggtattccattgcccaaattaacatggaccttcaataacaatattattccagcccactttgacagtgtgaatggacacagtgaacttgttattgaaagagtgtcaaaagaggattcaggtacttatgtgtgcaccgcagagaacagcgttggctttgtgaaggcaattggatttgtttatgtgaaagaacctccagtcttcaaaggtgattatccttctaactggattgaaccacttggtgggaatgcaatcctgaattgtgaggtgaaaggagaccccaccccaaccatccagtggaacagaaagggagtggatattgaaattagccacagaatccggcaactgggcaatggctccctggccatctatggcactgttaatgaagatgccggtgactatacatgtgtagctaccaatgaagctggggtggtggagcgcagcatgagtctgactctgcgaagtcctcctattatcactcttgagccagtggaaactgttattaatgctggtggcaaaatcatattgaattgtcaggcaactggagagcctcaaccaaccattacatggtcccgtcaagggcactctatttcctgggatgaccgggttaacgtgttgtccaacaactcattatatattgctgatgctcagaaagaagatacctctgaatttgaatgtgttgctcgaaacttaatgggttctgtccttgtcagagtgccagtcatagtccaggttcatggtggattttcccagtggtctgcatggagagcctgcagtgtcacctgtggaaaaggcatccaaaagaggagtcgtctgtgcaaccagccccttccagccaatggtgggaagccctgccaaggttcagatttggaaatgcgaaactgtcaaaataagccttgtccagtggatggtagctggtcggaatggagtctttgggaagaatgcacaaggagctgtggacgcggcaaccaaaccaggaccaggacttgcaataatccatcagttcagcatggtgggcggccatgtgaagggaatgctgtggaaataattatgtgcaacattaggccttgcccagttcatggagcatggagcgcttggcagccttggggaacatgcagcgaaagttgtgggaaaggtactcagacaagagcaagactttgtaataacccaccaccagcgtttggtgggtcctactgtgatggagcagaaacacagatgcaagtttgcaatgaaagaaattgtccaattcatggcaagtgggcgacttgggccagttggagtgcctgttctgtgtcatgtggaggaggtgccagacagagaacaaggggctgctccgaccctgtgccccagtatggaggaaggaaatgcgaagggagtgatgtccagagtgatttttgcaacagtgacccttgcccaacccatggtaactggagtccttggagtggctggggaacatgcagccggacgtgtaacggagggcagatgcggcggtaccgcacatgtgataaccctcctccctccaatgggggaagagcttgtgggggaccagactcccagatccagaggtgcaacactgacatgtgtcctgtggatggaagttggggaagctggcatagttggagccagtgctctgcctcctgtggaggaggtgaaaagactcggaagcggctgtgcgaccatcctgtgccagttaaaggtggccgtccctgtcccggagacactactcaggtgaccaggtgcaatgtacaagcatgtccaggtgggccccagcgagccagaggaagtgttattggaaatattaatgatgttgaatttggaattgctttccttaatgccacaataactgatagccctaactctgatactagaataatacgtgccaaaattaccaatgtacctcgtagtcttggttcagcaatgagaaagatagtttctattctaaatcccatttattggacaacagcaaaggaaataggagaagcagtcaatggctttaccctcaccaatgcagtcttcaaaagagaaactcaagtggaatttgcaactggagaaatcttgcagatgagtcatattgcccggggcttggattccgatggttctttgctgctagatatcgttgtgagtggctatgtcctacagcttcagtcacctgctgaagtcactgtaaaggattacacagaggactacattcaaacaggtcctgggcagctgtacgcctactcaacccggctgttcaccattgatggcatcagcatcccatacacatggaaccacaccgttttctatgatcaggcacagggaagaatgcctttcttggttgaaacacttcatgcatcctctgtggaatctgactataaccagatagaagagacactgggttttaaaattcatgcttcaatatccaaaggagatcgcagtaatcagtgcccctccgggtttaccttagactcagttggacctttttgtgctgatgaggatgaatgtgcagcagggaatccctgctcccatagctgccacaatgccatggggacttactactgctcctgccctaaaggcctcaccatagctgcagatggaagaacttgtcaagatattgatgagtgtgctttgggtaggcatacctgccacgctggtcaggactgtgacaatacgattggatcttatcgctgtgtggtccgttgtggaagtggctttcgaagaacctctgatgggctgagttgtcaagatattaatgaatgtcaagaatccagccctgtcaccagcgctgtttcaatgccataggaagtttccattgtggatgtgaacctgggtatcagctcaaaggcagaaaatgcatggattgt